(19) United States
(12) Patent Application Publication
(10) Pub. No.: US 2007/0077238 A1
(43) Pub. Date: Apr. 5, 2007

(54) Production of organic and inorganic selenium compounds by lactic acid bacteria

(76) Inventors: Alex Yeow-Lim Teo, Singapore (SG); Sook-Mei Hon, Johor Bahru (MY); Chea-Yun Se, Pontian Johor (MY); Hai Meng Ian, Singapore (SG)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(21) Appl. No.: 11/243,391

(22) Filed: Oct. 4, 2005

(51) Int. Cl. A61K 35/74; C12N 1/20

(52) U.S. Cl. 424/93.45; 424/780; 435/252.3

(57) ABSTRACT

A novel strain of lactic acid bacteria was found to be heat resistant and able to grow in a sulfur-limiting medium (SLM) containing a high concentration of sodium selenite. The microorganism is a non-spore forming and Gram-positive coccus, which is identified with >90% confidence using the API biochemical and sugar fermentation tests, ribotyoing and 16S rRNA sequencing as Pediococcus pentosaceus SP80. In the current study, P. pentosaceus SP80 grown on SLM containing 250 ppm sodium selenite produced both organic and inorganic forms of selenium. These selenium compounds can be separated using an anion exchange chromatography technique. The concentrations of selenium detected in the organic and inorganic fractions were 4.34 and 21.7 ppm, respectively. Selenium-enriched bacteria are useful as a source of selenium for supplementing the diets of animals and humans. Animals fed efficacious amounts of the selenium-enriched bacteria show improved feed conversion rates and higher levels of glutathione peroxidase (GPx) activity in heart, kidney and liver tissues indicating an increased absorption and retention of selenium over control diets.

### BACKGROUND OF THE INVENTION

The invention relates generally to the production of selenium compounds by bacteria and, more specifically, to the production of both organic and inorganic selenium compounds by lactic acid bacteria, and specifically bacteria of Pediococcus sp.

Selenium (Se) is an essential trace element discovered by the Swedish chemist, Jöns Jakob Berzelius in 1817. Similar to sulfur, selenium belongs to the group IV of the periodic table. It has six naturally occurring isotopes, namely ⁷⁴Se, ⁷⁶Se, ⁷⁷Se, ⁷⁸Se, ⁸⁰Se and ⁸²Se. The element exists in four oxidation states; −2, 0, +4 and +6. The oxidation state −2 is present in hydrogen selenide (H₂Se), a highly toxic gas that is readily oxidized to elemental selenium in the presence of air. Elemental selenium (Se⁰) and its compounds are highly soluble in water and soil, but can be toxic to biological systems at the level of parts per million (ppm). In terms of physical appearance, elemental selenium can exist in gray-black form of metallic hexagonal selenium and in an amorphous white or as monoclinic red (Se₈) forms. Inorganic selenium compounds include selenic acid (H₂SeO₄) and selenious acid (H₂SeO₃), at the +6 and +4 oxidative states, respectively. Both selenates (SeO₄⁻²) and selenites (SeO₃⁻²) have been reported to be more toxic than the organic selenium compounds. Organic seleno-compounds exist mainly as selenomethionine (SeMet), selenosystine (CySeSeCy) and selenocysteine (SeCys). Biologically, selenium species can be categorized at the enzymatic or genetic level. Selenocysteine is incorporated into the peptide backbone of glutathione peroxidase (GSH-Px), while SeMet replaces methionine in a random manner in amino acid translation. Seleno-amino acids, therefore, contain selenium in the form of selenocysteinyl, selenocystinal, and selenomethionyl residues.

Selenium is of fundamental importance to human and animal health. Humans can derive selenium from both plant and animal foodstuffs, while animals typically obtain the mineral via yeasts, corn and other plant sources. Absorption of selenium occurs mainly at the duodenal, caecal and colonic segments of the intestinal tract and is largely accumulated in the pancreas, pituitary glands and livers of mammals. Animals with selenium deficiency may develop symptoms, such as white muscle disease (WMD) and anemia in cattle and sheep; liver failure, WMD and mulberry heart in swine. Poultry may develop exudative diathesis, reduced growth rate and even death due to lack of selenium in their diets. Selenium also plays vital role in enhancing the immune system of animals and human. For sows fed with selenium-enriched diet, the litter size can be increased. Selenium supplementation during lactation increased the level of total selenium present in milk. In poultry, selenium can improve feather development, increase egg production and improve hatchability. The increased level of selenium could decrease drip loss and improve meat quality of broilers. However, acute toxicity of selenium can also occur at the concentration of between 5 and 25 mg/kg in the animal diets. Chronic selenium toxicity in animals lead to “blind stagger” and “alkali” disease.

The current-Recommended Daily Allowances (RDA) for selenium, established by the Food and Nutrition Board of the National Academy of Sciences (NAS), is 55 μg/day for both male and female adults. Selenium has also been regarded as a high nutritional value and act as antioxidant. The biochemistry of selenium is a complex system that enhances the immune system involves in inflammatory mechanisms that may be linked to diseases such as heart failure, cancer and rheumatoid arthritis in humans (1). Selenium deficiency in human was first reported as the Keshan disease, a type of heart disease called cardiomyopathy that affects young women and children in China. The disease has also been reported in New Zealand and Finland in areas where selenium in the soil is low (2). Although excessive intake of selenium can cause adverse health effects, these are generally observed at doses more than 5 times greater than the RDA.

Schwarz and Foltz found three agents that prevented liver necrosis in rats: vitamin E, cysteine and selenium. The enzyme glutathione peroxidase (GPx) was discovered as a selenium-dependent enzyme (3) and selenocysteine (SeCys) was found to be incorporated into peptide backbone of GPx as an essential component for the catalytic activities of selenium-dependent enzymes (4, 5). The main function of GPx is to protect hemoglobin against oxidative damage by hydrogen peroxide (5-10). The first type is GPx1, of 22 kDa and can be found in many tissues. GPx2 is mainly found in gastrointestinal tract and liver. Plasma GPx (GPx3) can be detected in kidney, heart and lung of mammals, while GPx4 is a phospholipid hydroperoxide found in many tissues. GPx is reported to protect mice from viral-induced myocarditis (11) and benign coxsackievirus in heart muscle in host mice (12). The other types of selenoproteins containing SeCys are iodothyronine-5′-deiodinase (type I and II), which is found in liver and kidney, thioredoxin reductase, Sel-W and Se-P. The selenoprotein Sel-W is a component of WMD that protects animal from the disease. Selenoprotein P, although with unknown function, is an extracellular glycoprotein that contains most of the selenium in plasma. The immune system in a host also has an effect on the association between viral diseases and nutrition. Dietary selenium plays a critical role and could also reduce oxidative stress that lead to pathogenesis of several viral infections, such as hepatitis and influenza (13).

Selenoproteins can be produced via the metabolism of selenium by various organisms. Organic and inorganic selenium may be concentrated in the biomass of plants, mushrooms, yeasts, fungus, Escherichia coli, and Lactobacillus spp. grown in media containing selenite or selenate. Many studies have also reported that selenium-containing compounds may readily enter the pathway of sulfur metabolism, and that O-acetylserine sulphydrylase is likely to be the entry site in Escherichia coli. Selenite metabolism has also been observed in the reduction of selenite-to elemental selenium and the incorporation of selenium into organic molecular structures.

A previous study by Turner et al. has shown that selenate and selenite may have entered the. cells of E. coli through the sulfate permease system (cysA, cysU, cysW) (14). However, only the synthesis of selenate was inhibited when the system is repressed suggesting the uptake of selenite by bacterial cells could be via an undefined carrier. They also reported that selenate exhibits a lower affinity for the sulfate permease pathway and its uptake is repressed by the presence of cysteine. Two organisms, Selenomonas ruminantium and Cldstridium pasteurianum, were discovered to have difficulty in transporting and metabolizing sulfate or selenate (15). Non-specific uptake rates for selenite and selenate by the sulfate permease in different bacterial species may be due to the differences in the kinetics of uptake between species (15). Selenite was believed to be bound to a protein through the vicinal thiol groups and released in the form of metallic selenium after accepting four electrons (16). Nickerson and Falcone suggested that the thiol content of protein could have determined the amount of selenite bound. The studies suggested that arsenite inhibition of the selenite reduction is not reversed by a monothiol substance and that selenite rapidly binds to SH-group in a divalent manner to form —S—Se—S linkages (16). Tomei et al. demonstrated that the transformation of selenate and selenite to elemental Se by Desulfovibrio desulfuricans occurred intracellularly when grown in media containing formate as the electron donor and either fumarate or sulfate as the electron acceptor (17).

Size exclusion and cation or anion exchange chromatography are common techniques used for the separation of various selenoamino acids (18-21). Inductively coupled plasma-mass spectrometry (ICP-MS) is a reliable technique that can detect sensitively most elements, inclusive of trace metals (18, 22-26). The ICP-MS technique has a detection limit of parts per trillion (ppt) or less with a 98-percent confidence level, established by several analytical laboratories (23). Various modular configurations involving the use of ICP-MS include quadrupole mass spectrometer, vacuum system or detector with liquid chromatography, high performance liquid chromatography, gas chromatography and/or mass spectrometry (LC-/HPLC-/GC-MS) have been used to study Se-enriched yeasts (18, 23-27).

Inorganic selenium (selenates and selenites) and selenium-enriched yeasts are commercially available as feed supplements in animal diets. Numerous types of selenium-yeast products consisting of varying quantities of organic and inorganic forms of selenium have been marketed in the feed industry (28). Studies have shown that the concentration of SeMet in selenium-yeast was found to range from 23% to 63%. However, the detection of SeMet in commercially available selenium-enriched yeast, Sel-Plex® (Alltech, Inc., USA), using HPLC was reported to involve the use of cyanogen bromide, which generates hydrogen cyanide.

Lactic acid bacteria (LAB) are naturally found in the gastrointestinal tract of human and animals. It is widely believed that they prevent the growth of putrefactive microorganisms in the gut system. Lactic acid bacteria is a group of Gram positive, non-pathogenic bacteria that can ferment carbohydrates to produce lactic acid. These bacteria can also possess proteolytic, lipolytic and β-galactosidase activities. Genera of LAB include Lactococcus, Lactobacillus, Streptococcus and Pediococcus. Lactobacillus acidophilus has been mixed with spray-dried selenium-enriched yeast products as supplements for the equine (29). Studies using sodium dodecyl sulfate-polyacrylamide gel electrophoresis (SDS-PAGE) technique have shown that selenium can become incorporated into bacterial proteins of L. delbrueckii subsp. bulgaricus (30, 31). However, no study to date has demonstrated the ability of Pediococcus spp. to take up and convert inorganic selenium into organic forms.

### SUMMARY OF THE INVENTION

The invention consists of a novel strain of bacteria identified herein as SP80, a non-spore forming and Gram-positive coccus, which is identified with API biochemical and sugar fermentation tests (97.3-99.9% confidence), ribotyping (83% confidence) and 16S rRNA sequencing (99.52%) as Pediococcus pentosaceus. Bacteria of the strain SP80 grow in a. sulfur-limiting medium that has been supplemented up to at least 1000 ppm with inorganic selenium compounds, such as sodium selenite. Analysis of the cell-free extracts of the bacteria showed an uptake of selenium and the presence of both inorganic and organic selenium compounds.

The selenium-enriched bacteria are fed to animals as a source of selenium. In a preferred embodiment, mice fed control diets including a selenium-depleted diet, a diet enriched with inorganic selenium and a diet supplemented with selenium-enriched yeast, were compared with mice fed a diet including the selenium-enriched bacteria. The mice fed the selenium-enriched bacteria showed an improved feed conversion rate and enhanced Glutathione Peroxidase (GPx) activity in heart, kidney and liver tissues, indicating a higher level of absorption and retention of selenium in these tissues.

### BRIEF DESCRIPTION OF THE FIGURE

FIG. 1 is a chart of selenium detection in parts per million (ppm) analyzed by inductively coupled plasma-mass spectrometry (ICP-MS); cell free extracts of the bacterial strain SP80 grown in sulfur-limiting medium containing 250 ppm of sodium-selenite were subjected to anion exchange chromatography for separation of organic and inorganic selenium

### DETAILED DESCRIPTION OF THE PREFERRED EMBODIMENTS

Inorganic selenium compounds, as used in this disclosure, include salts of selenium, and preferably alkali-metal salts of selenite and selenate, and more preferably sodium selenite.

Organic selenium compounds include seleno-amino acid compounds and complexes, and preferably selenomethionine, selenocystine, selenocysteine, Se-methylselenocysteine, gamma-glutamyl-Se-methylselenocysteine, gamma-glutamyl-selenomethionine, selenocystathionine and Se-adenosyl selenohomocysteine.

Lactic acid bacteria is a group of Gram positive, non-pathogenic bacteria that can ferment carbohydrates to produce lactic acid. Genera of LAB include Lactococcus, Lactobacillus, Streptococcus and Pediococcus.

### EXAMPLE 1

### Isolation and Identification of Lactic Acid Bacteria from the GI Tract of Chickens

Materials and Methods

Gastrointestinal tracts from three healthy chickens were obtained from a local market certified by the Agri-Food & Veterinary Authority of Singapore. Sections comprising the duodenum, jejunum and ileum of each intestinal tract were macerated and their contents were inoculated into de Man Rogosa Sharpe broth pH 6.3 (MRS) (Becton, Dickinson, USA) and incubated in an incubator set at 30° C. under 5% CO₂ for 24 h. Overnight cultures were streaked for isolation of pure colonies on MRS agar, pH 6.3 before sub-culturing in MRS broth using the same conditions and kept in 40% glycerol at −80° C. for long-term storage. Gram staining and biochemical tests (API® 50 CH test kit; bioMerieux, USA) were performed to identify the strains of lactic acid bacteria isolated from the gastrointestinal tract of healthy chickens.

Results

A total of 120 strains of microaerophilic and anaerobic bacteria were isolated from the duodenum, jejunum and ileum portions of the intestinal tract of healthy chicken using the MRS media, pH 6.5. Using the Gram-staining technique and biochemical tests, the morphologies and identities of these bacteria were identified. It was found that 97 strains of the bacteria isolated were Gram-positive bacteria with the majority showing a rod-shape morphology (Table 1).

TABLE 1
Isolation of lactic acid bacteria^{a} from gastrointestinal tracts of chicken

| Number of | | | |
|---|---|---|---|
| Gram- | Origin of isolation and morphology | | |
| positive | Duodenum | Jejunum | Ileum |

| strains | Rod | Cocci | Rod | Cocci | Rod | Cocci |
|---|---|---|---|---|---|---|
| 97 | 17 | 7 | 19 | 11 | 37 | 6 |

^{a}A total of 120 strains of bacteria were isolated from the gastrointestinal tract of healthy chicken. Out of these, 23 strains were Gram-negative bacteria.

### EXAMPLE 2

### Identification of Lactic Acid Bacteria (LAB) Strain SP80

Materials and Methods

A strain of LAB, identified herein as SP80 was selected out of the 97 strains previously isolated and screened for growth in media containing sodium selenite (25, 50, 100 and 200 ppm). The bacterium was selected based on the growth rate and ability of the cells to convert sodium selenite to amorphous selenium, indicated by the formation of red-pigmented colonies.

SP80 was grown in MRS broth at 30° C. under 5% CO₂ for 24 h. Five-ml portions of the culture were centrifuged at 5700×g for 10 min and the supernatant discarded. The pellet was then resuspended in CHL media (bioMérieux, USA) before dispensing into the cupules of the API® 50 CH test strips. The strips were incubated at 37° C. for 48 h. After 48 h, the results of the biochemical tests were analyzed using the automated identification software, API® LAB Plus™ (bioMérieux, USA). This strain was also found to be heat resistant at 60° C. for 30 min (data not shown). Results of the characterization study indicated that the bacterium has 97.3-99.9 percent identity with Pediococcus pentosaceus (Table 2).

TABLE 2
Biochemical and sugar fermentation profile of Pediococcus
pentosaceus SP80 by API ® 50 CH

| Sugars/Chemicals | Percentage (%) of positive reactions |
|---|---|
| Cellobiose | 100 |
| Esculin | 100 |
| Fructose | 100 |
| Galactose | 100 |
| Glucose | 100 |
| Glycogen | 100 |
| N-acetyl-glucosamine | 100 |
| Ribose | 100 |
| Salicin | 100 |
| Amygdalin | 99 |
| Arbutin | 99 |
| Maltose | 99 |
| Trehalose | 99 |
| Mannose | 92 |
| L arabinose | 83 |
| D tagatose | 67 |
| Dxylose | 42 |
| Rhamnose | 33 |
| Inulin | 8 |
| 2-keto-gluconate | 1 |
| Lactose | 1 |
| Melibiose | 1 |
| Raffinose | 1 |
| Sucrose | 1 |

The strain of Pediococcus pentosaceus SP80 isolated in our laboratory fermented nine different types of sugars, including amydalin, arbutin, maltose and trehalose. Consistent with the report by Facklam et al., (1989) P. pentosaceus was shown to react with salicin, maltose and trehalose (32). Our study indicated that P. pentosaceus SP80 is a Gram-positive non-spore forming coccus that grows well under microaerophilic conditions at 30° C. Samples of P. pentosaceus SP80 have been deposited with the American Type Culture Collection (ATCC) under accession number PTA 6736.

### EXAMPLE 3

### Uptake of Selenium by Pediococcus Pentosaceus SP80

Materials and Methods

Culture of Pediococcus pentosaceus SP80 on media containing sodium selenate and selenite. Pediococcus pentosaceus SP80 obtained from −80° C. freezer was resuscitated and grown in MRS broth at 30° C. under 5% CO₂ for 24 h. The cells were then inoculated into sulfur-limiting media (SLM; pH 6.0) containing 40 g/L of buffered peptone water (BPW; pH 7.4), 32 g/L Lab-Lemco powder, 16 g/L yeast extract (Oxoid, UK), 80 g/L glucose, 20 g/L sodium acetate trihydrant, 8 g/L di-potassium hydrogen phosphate anhydrous, 0.8 g/L magnesium chloride, 0.2 g/L manganese (II) chloride (Merck, Germany), 8 g/L citric acid and 4 g/L Tween® 80. The medium was then supplemented with sodium selenate (0, 250 ppm) or selenite (0, 1, 10, 50, 100, 250, 500 and 1000 ppm) (Sigma Chemicals, USA). The cultures were incubated at 30° C. under 5 % CO₂ for 24 h. Serial dilutions and plate count were performed using BPW and MRS agar, respectively.

Separation of organic and inorganic selenium by anion exchange chromatography. Overnight cultures containing Pediococcus pentosaceus SP80 were centrifuged at 20,000×g for 15 min and washed with phosphate buffered saline (PBS-1×; pH 7.4). The cell pellet containing SP80 was then resuspended in PBS-1× before disruption using a cell-sonicator (Misonix, USA) set at 6 W with 30-sec intervals for 15 cycles. Cell debris was treated with 1.5 M nitric acid (Merck, Germany) at 80° C. for 20 min. Cell free extracts were then obtained after being spun at 1000×g for 15 min. The pH of cell free extract was adjusted to 5.0-5.5 using 1 M sodium hydroxide (Merck, Germany). One-ml portion of the cell free extract was dispensed into a 1-cm (diameter)×20 cm (height) anion exchanger column (BIO-RAD, USA) packed with Dowex® 1-8 X (Sigma Chemicals, St Louis), with particle size of 100-200 mesh, to a height of 10 cm. Elution was performed with 0.01 M of sodium chloride and hydrochloric acid at 0.2 and 0.5 M, respectively. Five-ml fractions of the eluant were collected for Se analysis using ICP-MS.

Detection of total selenium by ICP-MS. An Elan 6100 ICP-MS (Perkin Elmer, USA) at PSB Corporation, Singapore was stabilized for one hour prior to injection of samples. A 3-point standard calibration was performed using the atomic spectroscopy standard at 10, 20 and 50 ppb, respectively. The atomic spectroscopy standard contains 10 ppb each of manganese, copper, rhodium, cadmium, indium, barium, lead and uranium. The accepted correction coefficient was set at less than 0.995. Five-ml portions of samples were then subjected to the Elan 6100 for ICP-MS analysis.

Results and Discussion

Putative strains of LAB were grown on MRS agar plates containing various concentrations of selenate and selenite. Strains that grew on MRS agar plates containing sodium selenate (25, 50, 100 and 200 ppm) formed white colonies, similar to those grown on control medium. In contrast, red-pigmented colonies were observed on agar media containing sodium selenite of up to 200 ppm. Generally, the color intensity of the colonies ranged from pink to dark red, corresponding with the increase in concentrations of sodium selenite used in the media. The coloration of colonies observed in the LAB cells grown on media containing sodium selenite may be due to the presence of amorphous selenium deposited by the bacteria. Gharieb (1995) has demonstrated that the reduction of selenite to elemental selenium on Czapek-Dox agar has resulted in the red-pigmented fungi colonies (33). Candida albicans was shown to rapidly reduce selenite but this reduction was inhibited by methionine, formate, fluoride, dinitrophenol and certain sulfhydryl poisons present in the medium (34).

Our screening experiments have demonstrated that some strains of the isolated 97 LAB were not inhibited by sodium selenite of up to 200 ppm. In addition, a number of these strains were found to be fast growers, with their colonies appearing only after 12 h of incubation (Table 3).

TABLE 3
Number of lactic acid bacteria strains^{a} grown
on media containing selenate or selenite

| | |
|---|---|
| Media supplements | Incubation time (h) |

| Selenium compounds^{b} | 12 | 24 | 48 | >48 |
|---|---|---|---|---|
| Selenate | 75.3 | 14.4 | 10.3 | 0 |
| Selenite | 62.9 | 2.1 | 24.7 | 10.3 |

^{a}percentage of bacterial cells that grew
^{b}250 ppm of selenate or selenite in media

The strain of Pediococcus pentosaceus identified herein as SP80 also grows well in a sulfur-limiting medium (SLM) containing up to 1000 ppm of selenite and produces a brick red pigmented culture when compared to those grown in selenate-enriched media. We suspected that SP80 may have metabolized sodium selenite and portions of which were converted to elemental Se. It is possible that excess elemental Se may then be deposited to the exterior of the cells and in the media as brick-red pigment. The reduction of selenite or selenate into elemental Se by microorganisms that causes the appearance of brick red pigments in the culture media were also observed by various researchers (14, 30, 31).

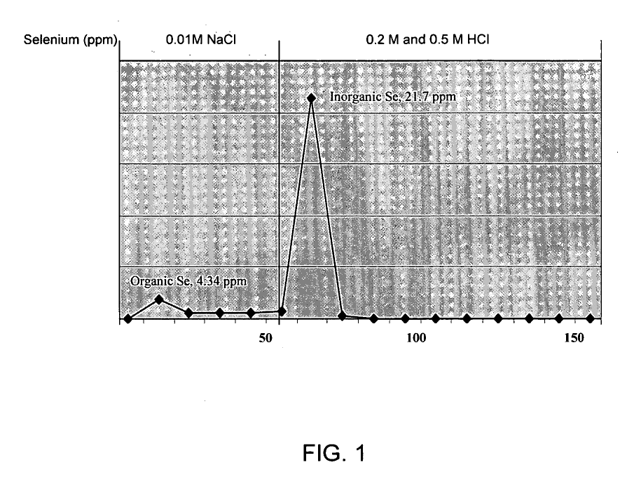

In our current study, selenium was detected in the cell free extract of the HNO₃-digested SP80 cells grown in SLM supplemented with 250 ppm of sodium selenite. Using ICP-MS technique, 32.20 ppm of total selenium was detected from hydrolyzed cells of SP80. No selenium was detected in the cell free extract of SP80 grown in the control media. When the cell free extract of selenium-enriched SP80 was passed through an anion exchanger, two organic and inorganic selenium fractions were eluted separately and collected. The fractions were subjected to analysis by ICP-MS and the results of this study indicated that the organic and inorganic fractions contained 4.34 and 21.7 ppm of selenium, respectively (FIG. 1). Zhang and Frankenberger had demonstrated the separation of selenium species from plant water extracts using the anion exchange chromatography (21). At pH 5-5.5, SeMet exists as zwitterionic ions and could be easily eluted out from the column packed with Dowex® by 0.01 M of NaCl. In contrast, the inorganic selenium remained bound to the resin and could only be eluted using acidic (0.2 and 0.5M of HCl, respectively) solutions.

Accordingly, a novel strain of lactic acid bacteria, strain SP80 was able to grow in a sulfur-limiting medium containing high concentrations of selenate and selenite. The strain has been identified using biochemical and sugar fermentation tests as Pediococcus pentosaceus. Pediococcus pentosaceus SP80 when cultured in a sulfur-limiting medium was found to take up selenite, convert or reduce it to elemental Se, forming a brick-red precipitation. The organic and inorganic selenium present in P. pentosaceus SP80 were fractionated using an anion exchange chromatography technique. The selenium content in both organic and inorganic fractions was analyzed and quantified using ICP-MS analysis. The selenium concentrations in the organic and inorganic fractions were found to be 4.34 and 21.7 ppm, respectively.

### EXAMPLE 4

### In Vivo Testing

Materials and Methods

Bacterial Culture and Culture Conditions. The pure culture of Pediococcus pentosaceus SP80 was resuscitated and grown in a sulfur-limiting medium (SLM; pH 6) supplemented with 10 ppm of sodium selenite (Sigma Chemicals, USA). The bacteria were grown at 30° C. under 5% CO₂ condition for 24 h.

Scale-Up Fermentation and Freeze Drying of Bacterial Culture. A 2-L B-Braun Biostat® B-DCU Stirred Tank Fermenter (Braun, Germany) was used to produce 10 liters of fermented culture containing approximately 10⁸ CPU per ml of P. pentosaceus SP80. The medium was sterilized in the fermenter by autoclaving at 121° C. for 20 min. After the medium has cooled to room temperature, a 1-percent overnight seed culture of P. pentosaceus SP80 was inoculated into the pre-sterilized sulfur-limiting medium containing 10 ppm of sodium selenite. The fermentation temperature was maintained at 30° C. The culture was allowed to grow in the fermenter for 24 h. The 24-h culture was then freeze-dried at −40° C. under vacuum.

Design of Trial

Preparation of mice feed supplements and diets. The mice feed was obtained from a local feed importer. The composition of the mice feed is set out in Table 4. The mice pellets were ground and passed through a mesh-20 size sieve.

TABLE 4
The composition and calculated analysis of the basal diets

| | Feed Diet | |
|---|---|---|
| Ingredients | Added vitamins and minerals per kg | |
| Vitamin A | 10 000 | IU |
| Vitamin B₁₂ | 30 | mg |
| Vitamin D | 2 000 | IU |
| Vitamin E | 100 | mg |
| Vitamin K | 2.0 | mg |
| Nicotinic acid | 25 | mg |
| Folic acid | 2.0 | mg |
| Thiamine | 8.0 | mg |
| Pyridoxine | 8.0 | mg |
| Copper | 16 | mg |
| Magnesium | 100 | mg |
| Molybdenum | 0.5 | mg |
| Selenium | 0.1 | mg |
| Riboflavin | 8 | mg |
| Biotin | 100 | mg |
| Iodine | 0.5 | mg |
| Iron | 70 | mg |
| Manganese | 70 | mg |
| Zinc | 80 | m |

Calculated analysis

| | |
|---|---|
| Digestible Energy | 14.3 MJ/Kg |
| Protein, % | 19.0 |
| Fat, % | 4.5 |
| Crude fiber, % | 4.5 |
| Calcium, % | 0.77 |
| Phosphorus, % | 0.57 |
| Salt, % | 0.41 |

Experimental feed A is the negative control diet containing ground feed alone (Table 5). Experimental feeds B, C, and D are negative control diets supplemented with sodium selenite, freeze-dried SP80 prepared and described as before, and Sel-Plex®, a commercial source of selenium-enriched yeast (Alltech, USA), respectively (Table 5). Except for the negative control diet, the final concentrations of total selenium in experimental feeds B, C and D were adjusted to 0.3 ppm, respectively.

TABLE 5
Experimental Design

| Group | Treatment diet | Male | Female | Total |
|---|---|---|---|---|
| A | Se-depleted mouse feed | 4 | 4 | 8 |
| | (negative control) | | | |
| B | Sodium selenite-fortified feed | 4 | 4 | 8 |
| C | Se-rich bacteria-fortified feed (Kemin) | 4 | 4 | 8 |
| D | Se-rich yeast-fortified feed (SelPlex) | 4 | 4 | 8 |
| | Total | 16 | 16 | 32 |

Pre-treatment of study animals. Thirty-two adult mice used in the experiment were divided into 4 treatment groups (Table 5). Each group consisted of 4 male and 4 female mice. Four mice of the same gender were kept in one cage. There was a short acclimatization period of 1 week before feeding them with the various diets. All mice received the control diet for 1 week before the intervention.

Animal management. All mice were kept at 25° C. in an incubator. Feed and water were available ad libitum. The weight of all mice and the feed consumed was monitored on a weekly basis throughout the period of study.

Glutathione Peroxidase Assay

On the sixth week, mice were randomly selected from each treatment group per cage to be sacrificed and their serum, heart, liver and kidney were obtained for analysis. Prior to dissection, tissues were perfused with 0.9% NaCl containing 0.16 mg/ml of heparin to remove red blood cells and clots. One-gram of tissue was then homogenized in 4-8 ml of cold buffer containing 50 mM Tris-HCl (pH 7.5), 5 mM EDTA, and 1 mM DTT. The suspension was then centrifuged at 10,000×g for 20 min at 4° C. After 20 min, the supernatant was removed for analysis using the Cellular Calibiochem®'s Glutathione Peroxidase Assay Kit (Cat# 354104) (Merck KGaA, Germany). The bioavailability of selenium was determined using a spectrophotometer at 340 nm to measure the glutathione peroxidase (GPx) activity in the serum, heart, liver and kidney of the mice from each treatment group. The calculation of GPx activity is determined by dividing the sum of the net rate of NADPH and the reduction X sample factor by 0.0062.

Analysis of Data

Results from the treated groups were-compared with the control groups and between treatments using the GraphPad Prism statistical package (GraphPad Software, Inc., USA) to test for significant difference at p<0.05. Body weight gains, feed intakes, feed conversion ratios and GPx activities were also subjected to analyses of variance and the standard errors of difference tested for statistical significance.

Analysis of the Results

In the current study, mice are fed on different diets containing organic and inorganic selenium from various sources. Regardless of their origins, the mineral, selenium has to be ingested, absorbed and retained within the mice in order for it to perform its function. Glutathione Peroxidase (GPx) is a tetrameric protein weighing approximately 85,000 D. GPx has 4 atoms of selenium bound as seleno-cysteine moieties that confer the catalytic activity. Therefore, the Glutathione Peroxidase assay was performed to compare the glutathione peroxidase activities between different treatment groups to reflect the level of Se absorbed and retained, in various tissues of mice fed with selenium from various sources. In our study, the GPx activity in various organs was consistently higher in male mice fed on diets containing Se-enriched bacteria when compared to the negative control diets (P<0.05) (Tables 7-9). Results from the mice trial also consistently showed that the GPx activity in livers, kidneys and hearts was higher in mice fed on diets containing Se-enriched bacteria compared to those that fed on Se-enriched yeast (P<0.05) (Tables 7-9). Our data also indicated that the activities of GPx within the liver and heart of female mice fed on diets containing Se-enriched bacteria were higher compared to the negative control diets (P<0.05) (Tables 8, 9). In terms of feed intake efficiency, it is interesting to note that although a standard 5-gram of the respective feed was given to each mouse per day, mice fed with Se-enriched bacteria showed a significant enhancement in feed conversion rate (FCR) of the male mice compared to those fed on negative control diets (P<0.005) (Table 6).

TABLE 6
Influence of selenium on the feed conversion ratio (FCR)
of male and female mice after 6 week of treatment

| | Male FCR | Female FCR |
|---|---|---|
| Treatment | [g/g] | [g/g] |
| Se-depleted mouse feed (negative control) | 81.79^{a} | 126.43^{a} |
| Sodium selenite-fortified feed | 71.00^{b} | 144.85^{ab} |
| Se-rich bacteria-fortified feed (SP80) | 60.16^{b} | 119.68^{ab} |
| Se-rich yeast-fortified feed (Sel-Plex) | 72.60^{ab} | 197.82^{b} |
| Significance, P= | 0.0272 | 0.0182 |

^{a,b}Values with different superscripts in the same column are significantly different [P < 0.05]; each mean value represents an average of 4 replicates of male or female mice per cage throughout 6 weeks of feeding.

TABLE 7
Influence of selenium on the feed GPx activity in the hearts
of male and female mice after 6 week of treatment

| | Male GPx | Female GPx |
|---|---|---|
| Treatment | [mU/ml] | [mU/ml] |
| Se-depleted mouse feed (negative control) | 763.36^{a} | 730.55^{a} |
| Sodium selenite-fortified feed | 688.532^{a} | 552.42^{b} |
| Se-rich bacteria-fortified feed (SP80) | 1195.28^{b} | 746.62^{a} |
| Se-rich yeast-fortified feed (Sel-Plex) | 684.67^{a} | 535.48^{b} |
| Significance, P= | 0.0068 | 0.0002 |

^{a,b}Values with different superscripts in the same column are significantly different [P < 0.05]; each mean value represents an average of 4 replicates of male or female mice per cage after 6 weeks of feeding.

TABLE 8
Influence of selenium on the feed GPx activity in the kidneys
of male and female mice after 6 week of treatment

| | Male GPx | Female GPx |
|---|---|---|
| Treatment | [mU/ml] | [mU/ml] |
| Se-depleted mouse feed (negative control) | 2879.74^{a} | 5713.18^{a} |
| Sodium selenite-fortified feed | 4308.68^{b} | 5872.67^{a} |
| Se-rich bacteria-fortified feed (SP80) | 5421.22^{c} | 7148.55^{b} |
| Se-rich yeast-fortified feed (Sel-Plex) | 4113.18^{b} | 5934.41^{a} |
| Significance, P= | <0.0001 | 0.0102 |

^{a,b}Values with different superscripts in the same column are significantly different [P < 0.05]; each mean value represents an average of 4 replicates of male or female mice per cage after 6 weeks of feeding.

TABLE 9
Influence of selenium on the feed GPx activity in the livers
of male and female mice after 6 week of treatment

| | Male GPx | Female GPx |
|---|---|---|
| Treatment | [mU/ml] | [mU/ml] |
| Se-depleted mouse feed (negative control) | 8850.16^{a} | 18492.60^{a} |
| Sodium selenite-fortified feed | 7859.81^{a} | 19200.86^{b} |
| Se-rich bacteria-fortified feed (SP80) | 11857.23^{b} | 20149.20^{b} |
| Se-rich yeast-fortified feed (Sel-Plex) | 8778.14^{a} | 19350.91^{b} |
| Significance, P= | 0.0024 | 0.0033 |

^{a,b}Values with different superscripts in the same column are significantly different [P < 0.05]; each mean value represents an average of 4 replicates of male or female mice per cage after 6 weeks of feeding.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

### REFERENCES

1. Arthur J R., McKenzie C., Beckett G J. (2003) Selenium in the Immune System, Journal of Nutrition; 133: 1457S-1459S.
2. Tan J., Zhu W., Wang W., Li R., Hou S., Wang D., Yang L. 2002. Selenium in soil and endemic diseases in China. Science of the Total Environment; 284(1-3): 227-235.5.
3. Schwarz K., Foltz C. M. 1957. Selenium as an integral part of factor 3 against dietary necrotic liver degeneration. Journal of the American Chemical Society; 79: 3292.
4. Zakowski J. J., Forstom J. W., Condell R. A., Tappel A. L. 1978 Attachement of selenocysteine in the catalyticsite of glutathione peroxidase, Biochemical and Biophysical Research Communications; 84: 248.
5. Birringer M., Pilawa S., Flohé L. 2002. Trends in selenium biochemistry. Nat Prod Rep; 19: 693-718.
6. Sunde R. A. Molecular Selenium Nutrition: A Journey from Rats to Molecular Biology. Nutritional Sciences and Biochemistry, University of Missouri, Columbia. For A. L. Moxon Honorary Lectures, Special Circular 167-199, The Ohio State University.
7. Arthur J. R. 2000. The glutathionine peroxidases. Cellular and Molecular Life Sciences; 57: 1825-1835.
8. Tanaka H., Esaki N., Soda K. 1985. Selenocysteine metabolism in mammals. Current Topics in Cellular Regulation; 27: 487-495.
9. Stadtman T. 1996. Selenocysteine. Annual Review of Biochemistry; 65: 83-100.
10. Forstrom J. W., Zakowski J. J., Tappel A. L. 1978. Identification of the catalytic site of rat liver glutathioneperoxidase as selenocysteine, Journal of Biochemistry; 17: 2639.
11. Beck M A., Esworthy R S., Ho Y S., Chu F F. (1998) Glutathione peroxidase protects mice from viral-induced myocarditis, FASEB J; 12: 1143-1149.
12. Beck M A., Shi Q., Morris V C., Levander O A. (2004) Benign coxsackievirus damages heart muscles in iron-loaded vitamin E-deficient mice, Free Radical Biology and Medicine; 38: 112-116.
13. Beck M A., Levander O A. (1998) Dietary oxidative stress and the potentiation of viral infection, Annual Review of Nutrition; 18: 93-116.
14. Turner R. J., Weiner J. H., Taylor D. E. (1998) Selenium metabolism in Escherichia coli, BioMetals; 11: 223-227.
15. Heider J. and Böck A. (1993) Selenium metabolism in micro-organisms, Advances in Microbial Physiology; 35: 71-107.
16. Nickerson W. J., Falcone G. (1963) Enzymatic reduction of selenite, Journal of Bacteriology; 85: 763-771.
17. Nickerson W. J., Falcone G. (1963) Enzymatic reduction of selenite, Journal of Bacteriology; 85: 763-771.
18. Uden, P. C., Bird S. M., Kotrebai M., Nolibos P., Tyson J. F., Block E. (1998) Analytical selenoamino acid studies by chromatography with interfaced atomic mass spectrometry and atomic emission spectral detection, Journal of Analytical Chemistry; 362: 447-456.
19. Cavalli S., Cardellicchio N. (1995) Direct determination of seleno-amino acids in biological tissues by anion-exchange separation and electrochemical detection, Journal of Chromatography A; 706: 429-436.
20. Davis W. C., Jin F., Dempster M. A., Robichaud J. L., Marcus R. K. (2002) Development of a new liquid chromatography method for the separation and speciation of organic and inorganic selenium compounds via particle beam-hollow cathode glow discharge-optical emmission spectroscopy, Journal of Analytical Atomic Spectrometry; 17: 99-103.
21. Zhang Y., Frankenberger. W. T. Jr. (2001) Speciation of selenium in plant water extracts by ion exchange chromatography—hydride generation atomic absorption spectrometry, The science of the Total Environment; 269: 39-47.
22. Laborda F., Vicente M. V., Mir J. M. Castillo J. R. (1997) Evaluation of on-line coupling size exclusion chromatography eletrothermal atomic absorption for selenium speciation, Journal of Analytical Chemistry; 357: 837-843.
23. The 30-minute guide to ICP-MS, (2001), http://www.perkinelmer.com.
24. Bird S. M., Uden P. C., Tyson F. T., Block E., Denoyer E. (1997) Speciation of selenoamino acids and organoselenium compounds in selenium-enriched yeast using HPLC-inductively coupled plasma mass spectrometry, Journal of Analytical Atomic Spectrometry; 12: 785-788.
25. Lindemann T., Hintelmann H. (2002) Identification of selenium-containing glutathione S-conjugate in a yeast extract by two-dimensional liquid chromatography with inductively coupled plasma MS and nanoelectrospray MS/MS detection, Analytical Chemistry; 74: 4602-4610.
26. Kotrebai M., Biringer M., Tyson J. F., Block E., Uden P. C. (1999) Identification of the principal selenium compounds in selenium-enriched natural sample extracts by ion-pair liquid chromatography with inductively coupled plasma- and electrospray ionization-mass spectrometric detection, Analytical Communications; 36: 249-252.
27. Ximénez-Embún P., Alonso I., Madrid—Albarran Y., Camara C., (2004) Establishment of selenium uptake and species distribution in Lupine, Indian mustard, and sunflower plants, Journal of Agricultural and Food Chemistry; 52: 832-838.
28. Ward T. (2003) Selenium yeast: Quality concerns, Asian Poultry Magazine; June: 32-
29. UBL Corp. Equine products international export division, http://www.cam.org
30. Calomme. M. R., Van den Branden K., Vanden Berghe D. A. (1995) Selenium and Lactobacillus species, Journal of Applied Bacteriology; 79: 331-340.
31. Calomme M., Hu J., Van Den Branden K. and Vanden Berghe D. A. (1995) Seleno-Lactobacillus an organic selenium source, Biological Trace Element Research; 47: 379-383.
32. Facklam R., Hollis D., Collins M. D. (1989) Identification of Gram-positive coccal and coccobacillary vancomycin-resistant bacteria, Journal of Clinical Microbiology; 27: 724-730.
33. Gharieb M. M. (1995) Reduction of selenium oxyanions by unicellular, polymorphic and filamentous fungi: cellular location of reduced selenium and implication for tolerance, Journal of Industrial Microbiology; 14: 300-311
34. Nickerson W. J., Falcone G. (1963) Enzymatic reduction of selenite, Journal of Bacteriology; 85: 763-771.

We claim:

1. An isolated bacterium identified as SP80 and deposited with the ATCC under accession number PTA 6736.

2. A process for providing a source of selenium to an animal, comprising the steps of growing lactic acid bacteria capable of uptaking selenium in a medium enriched with selenium and administering an effective amount of the selenium-enriched lactic acid bacteria or extracts or parts thereof to an animal.

3. A method as defined in claim 2, wherein the lactic acid bacteria comprise the genus Pediococcus.

4. A method as defined in claim 2, wherein the lactic acid bacteria comprise the strain Pediococcus pentosaceus SP80.

5. A method as defined in claim 2, wherein the medium is enriched with inorganic selenium compounds and the lactic acid bacteria are enriched with organic selenium compounds.

6. A method as defined in claim 5, wherein the inorganic selenium compounds are selected from the group consisting of selenite and selenate.

7. A method as defined in claim 5, wherein the organic selenium compounds are selected from the group consisting of selenomethionine, selenocystine, selenocysteine, Se-methylselenocysteine, gamma-glutamyl-Se-methylselenocysteine, gamma-glutamyl-selenomethionine, selenocystathionine and Se-adenosyl selenohomocysteine.

8. A method as defined in claim 5, wherein the lactic acid bacteria comprise the strain Pediococcus pentosaceus SP80.

9. A method of converting inorganic selenium compounds to organic selenium compounds, comprising feeding the inorganic selenium compounds to lactic acid bacteria capable of uptaking inorganic selenium and producing organic selenium compounds.

10. A method as defined in claim 9, wherein the inorganic selenium compounds are selected from the group consisting of selenite and selenate, and the organic selenium compounds are selected from the group consisting of selenomethionine, selenocystine, selenocysteine, Se-methylselenocysteine, gamma-glutamyl-Se-methylselenocysteine, gamma-glutamyl-selenomethionine, selenocystathionine and Se-adenosyl selenohomocysteine.

11. A method as defined in claim 9, wherein the lactic acid bacteria comprise the strain Pediococcus pentosaceus SP80.

12. A method of improving the feed conversion rate in an animal, comprising the step of feeding the animal an effective amount of lactic acid bacteria enriched with selenium.

13. A method of increasing the absorption and retention of selenium by an animal, comprising feeding the animal an effective amount of lactic acid bacteria enriched with selenium.

14. A process for providing a source of selenium to a human, comprising the steps of growing lactic acid bacteria capable of uptaking selenium in a medium enriched with selenium and administering an effective amount of the selenium-enriched lactic-acid bacteria or extracts or parts thereof to human.

15. A method as defined in claim 14, wherein the lactic acid bacteria comprise the genus Pediococcus.

16. A method as defined in claim 14, wherein the lactic acid bacteria comprise the strain Pediococcus pentosaceus SP80.

17. A method as defined in claim 14, wherein the medium is enriched with inorganic selenium compounds and the lactic acid bacteria are enriched with organic selenium compounds.

18. A method as defined in claim 14, wherein the inorganic selenium compounds are selected from the group consisting of selenite and selenate.

19. A method as defined in claim 14, wherein the organic selenium compounds are selected from the group consisting of selenomethionine, selenocystine, selenocysteine, Se-methylselenocysteine, gamma-glutamyl-Se-methylselenocysteine, gamma-glutamyl-selenomethionine, selenocystathionine and Se-adenosyl selenohomocysteine.

20. The use of Pediococcus pentosaceus SP80 as a source of organic selenium compounds for immune-related diseases.
